(19) United States
(12) Patent Application Publication
(10) Pub. No.: US 2021/0319878 A1
(43) Pub. Date: Oct. 14, 2021

(54) MEDICAL DETECTION SYSTEM AND METHOD

(71) Applicant: ElectrifAi, LLC, Jersey City, NJ (US)

(72) Inventor: Georgios Ouzounis, Weehawken, NJ (US)

(73) Assignee: ElectrifAi, LLC, Jersey City, NJ (US)

(21) Appl. No.: 17/162,398

(22) Filed: Jan. 29, 2021

Related U.S. Application Data
(60) Provisional application No. 63/009,685, filed on Apr. 14, 2020.

(51) Int. Cl. G16H 30/00; G06N 20/00; G06K 9/62; G06K 9/68

(52) U.S. Cl. CPC G16H 30/00; G06K 9/6807; G06K 9/6202; G06N 20/00

(57) ABSTRACT

A system and method, after performing the comparison, identifies at least one a match of the target feature from the imaging source with a library of target features. The system and method receive at least one numerical representation from a diagnostic source of a target feature. The system and method compares the at least one numerical representation of the target feature with a library of numerical representations. The system and method, after performing the comparison, identifies at least one match of the target feature from the imaging source with the library.

### BACKGROUND

### Field of the Disclosure

The present disclosure generally relates to medical examination equipment generally, and more particularly, to a system and method for examining hypodermic target features.

### Description of Related Art

This section intends to provide a background discussion for a clear understanding of the disclosure herein, but makes no claim nor any implication as to what is the relevant art for this disclosure.

Various medical examination equipment are currently employed for examining hypodermic, subdural and/or subcutaneous target features. For the purposes of the present disclosure, the phrase “target feature” is defined as any hypodermic organ, bone, tissue, circulatory or cell structure, such as, merely for exemplary purposes, lungs, the heart, liver, pelvis, pulmonary artery, spinal disk, joint cartilage or sciatic nerve, and any anomalies thereto, including thrombosis, tumors, the “ground-glass” patterns associated with pneumonia, apparent to skilled medical professionals. These instruments typically involve techniques including, but not limited to, X-rays, fluoroscopy, magnetic resonance, tomography, and ultrasound. Each of these approaches are adept at detecting anomalies in target feature such as bone, organ, tissue, circulatory structure, or tumors, for example, with varying degrees of efficacy to avert the need of exploratory surgery.

One area of particular interest, though without limitation or reservation, is Computerized Axial Tomography (CAT) scans, also known simply as CT scans. CT scans are a medical imaging modality that rely on two dimensional slices of a target feature(s) obtained from a large series of two-dimensional X-ray images taken in different directions. Each slice of a target feature can be reduced to numerical representations much like the pixels on a two dimensional monitor.

A problem with imaging technology generally, and two dimensional solutions particularly, is the detectability of the target feature. Not every static hypodermic, subdural and/or subcutaneous target feature is discernable to the naked human eye regardless of the number of CT scan slices taken of the specific area(s) of the body. The static hypodermic, subdural and/or subcutaneous target features are often occluded by other non-target features, obscuring them from analysis or visual inspection. This limitation poses issues for a medical practitioner in examining a range of target features including acute anomalies from tumors to pneumonia to COVID-19.

Methods for examining targeted features may include the application of convolution neural networks (“CNNs”) and Deep Neural Networks (“DNNs”). These methods are challenged by detailed geometric information about the target features. This is due to their inherent estimation, approximation, inference, blurring, and other effects that provide a crude approximation of the shape of the target feature.

### SUMMARY

The present disclosure includes a system and method for examining at least one target feature.

In one embodiment of the disclosure, the system and method receive at least one numerical representation, or data set, from an examination equipment source of a target feature. The system and method compares at least one data set of the target feature with a library or look-up table of numerical representations, or data sets, of representative target features. The system and method, after performing the comparison, identifies at least one closest match of the one or more data sets associated with the target feature from the examination equipment source with the library representative target features.

In another embodiment of the disclosure, at least one pixel group is created from the at least one data set of the target feature. For the purposes of the present disclosure, a pixel group is defined as a group of pixels, which may be a subset of the one or more data sets, which may be arranged in a hierarchal tree representation and, for exemplary purposes, may be characterized as lossy compressed data, lossless compressed data and/or vector attribute data.

In another embodiment of the disclosure, the library of data sets of representative target features may be, for exemplary purposes, characterized as lossy compressed data, lossless compressed data and/or vector attribute data.

In yet another embodiment of the disclosure, the system and method qualifies the one or more closest matches with a significance score of the one or more closest pixel group matches of the target feature with the library of data sets of representative target features.

In yet another embodiment, a vector attribute function is performed on the at least one data set of the target feature to create at least one set of vector attributes for the target feature.

In yet still another embodiment, the library includes vector attributed data and the comparing step is performed between each vector attributed target feature data set with the library of vector attributed data of representative target features.

In still another embodiment, each vector attribute created from at least one data set of the target feature is compared with a library of vector attribute data of representative target features to identify at least one closest match.

In another embodiment, the method includes the step of scanning each vector attribute from at least one data set of the target feature and computing at least one max-tree of at least two dimensions. For the purposes of the present disclosure, a max-tree is defined as a hierarchical representation of at least one image forming the basis of a large family of morphological filters. The one or more calculated max-tree computations are compared with each vector attribute stored in the library of representative target features. Each vector attribute may include an individual library to store the vector attributes derived from a series of data sets calculated from the target feature originating from a medical examination equipment source.

In yet another embodiment, the data sets created for the target feature are resealed to reduce computation time. Here, each vector attribute may be scale invariant and the comparison with the library of references of the target features may be realized using Euclidean distance measurements.

In yet still another embodiment, after the one or more closest data set matches of the target area with the library of representative target features is detected, a matching score is created and compared with a safety standard threshold. If the matching score(s) is higher than the safety standard threshold, the closest data set match(es) and/or the associated vector attribute(s) is or are stored in memory.

In yet another embodiment, a three dimensional max-tree is created from the one or more pixel groups created from the one or more data sets of the target feature, originating from the medical examination equipment source. Here, a three dimensional characterization of the target feature may be assembled to find at least one match between at least one closest pixel group and/or the associated vector attribute(s) stored in memory. With the match identified, a pixel to mapping segment function may be performed, allowing for the return other voxels. For the purposes of the present disclosure, a voxel represents a value on a regular grid in three-dimensional space. As with pixels in a two dimensional bitmap, voxels may have their position unencoded with their values though rendering systems may infer the position of a voxel based upon its position relative to other voxels.

### BRIEF DESCRIPTION OF THE DRAWINGS

The present disclosure and its various features and advantages can be understood by referring to the accompanying drawings by those skilled in the art relevant to this disclosure. Reference numerals and/or symbols are used in the drawings. The use of the same reference in different drawings indicates similar or identical components, devices or systems. Various other aspects of this disclosure, its benefits and advantages may be better understood from the present disclosure herein and the accompanying drawings described as follows:

FIG. 1 illustrates an embodiment of the present disclosure;

FIG. 2 illustrates an embodiment of the present disclosure;

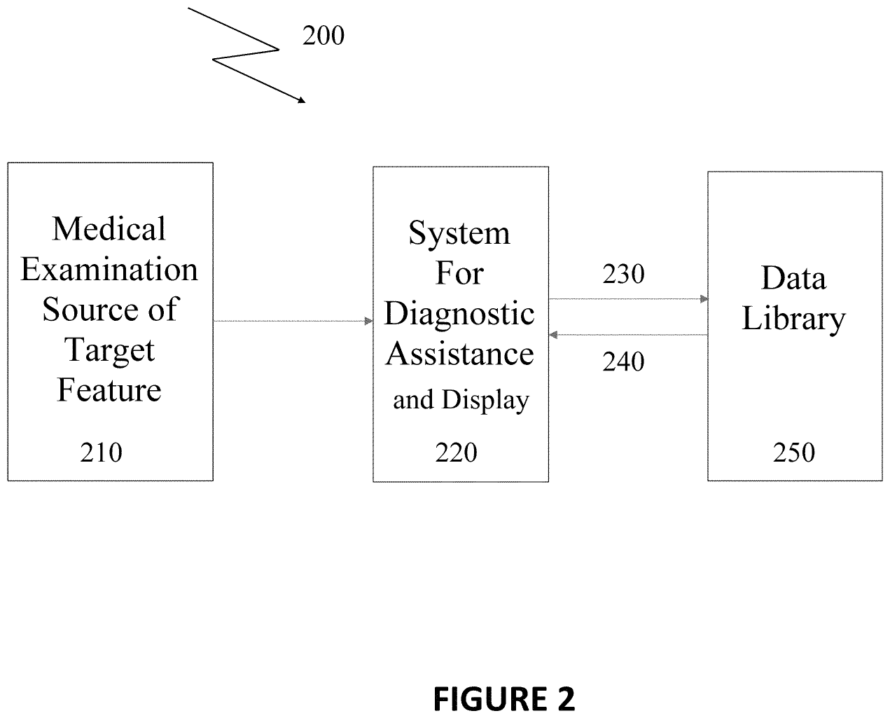

FIG. 3 illustrates another embodiment of the present disclosure;

FIG. 4 illustrates yet another embodiment of the present disclosure;

and

FIG. 5 illustrates yet another embodiment of the present disclosure.

### DETAILED DESCRIPTION

The present disclosure is a system and method for examining target features as defined hereinabove.

In one aspect of the disclosure, the system and method receive one or more numerical representations, or data set, from a medical examination equipment source of a target feature. The system and method compares the one or more data sets of the target feature with a library or look-up table of numerical representations, or data sets, of representative target features. The system and method, after performing the comparison, identifies one or more closest matches of the at least on data set associated with the target feature from the medical examination equipment source with the library representative target features. These closest match or matches are arrived by setting a quality threshold.

In another aspect of disclosure, one or more pixel groups, as defined hereinabove, are created from the one or more data sets of the target feature, while the library of data sets of representative target features may be, for exemplary purposes, characterized as lossy compressed data, lossless compressed data and/or vector attribute data. Here, a comparison of the one or more pixel groups is performed with the library of data sets, characterized in any format, to arrive at one or more closest matches. The system and method thereafter qualifies the one or more closest matches with a significance score.

In yet another aspect of disclosure, a vector attribute filtering function is performed on one or more data sets of the target feature to create one or more sets of vector attributes for the target feature, while the library of representative target features includes vector attributed data. Consequently, the system and method may compare each vector attribute(s) of the target feature(s) with that stored within the library.

In still yet another aspect of disclosure, the system and method includes the step of scanning each vector attribute from one or more data sets of the target feature to compute one or more max-tree, as defined hereinabove, of at least two dimensions. The one or more calculated max-tree computations are compared with each vector attribute stored in the library of representative target features. Each vector attribute may include an independent, target feature library to store the vector attributes derived from the series of data sets calculated from the target feature originating from the source medical examination equipment.

In still another aspect of the disclosure, the data sets created for the target feature are resealed to reduce computation time. Here, each vector attribute may be scale invariant and the comparison with the library of references of the target features may be realized using Euclidean distance measurements.

In yet still another aspect of the disclosure, after the one or more closest data set match between the target area and the library is computed, a matching score is created and compared with a safety standard threshold. If the matching score is higher than the safety standard threshold, the one or more closest data set matches and/or the associated vector attribute(s) Is flagged and may be stored in memory.

In yet another aspect of the disclosure, a three dimensional max-tree is created from one or more pixel groups created from the at least one data set of the target feature, originating from the medical examination equipment, such as an imaging source. Here, a three dimensional characterization of the target feature can be assembled to find a match of the one or more closest pixel groups and/or the associated vector attribute(s) stored in memory. With the match identified, a pixel to mapping segment function may be performed, allowing for the return all other voxels. For the purposes of the present disclosure, a voxel represents a value on a regular grid in three-dimensional space. As with pixels in a two dimensional bitmap, voxels typically do not have their position explicitly encoded with their values but rendering systems may infer the position of a voxel based upon its position relative to other voxels.

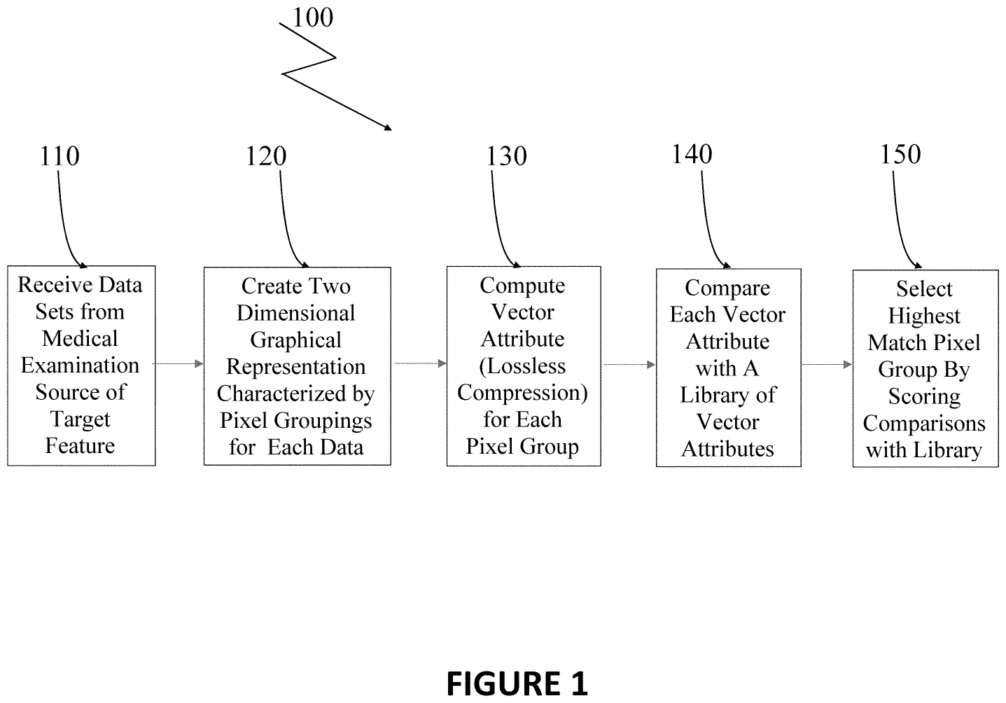

Referring to FIG. 1, a first embodiment of the present disclosure is illustrated. Here, a flow chart is depicted showing for a method 100 of examining one or more target features. As noted hereinabove, a target feature is defined as a data set representation of an area of focus by a medication professional that may include hypodermic organ, bone, tissue, circulatory or cell structure, such as, merely for exemplary purposes, lungs, the heart, liver, pelvis, pulmonary artery, spinal disk, joint cartilage or sciatic nerve, and any anomalies thereto, including thrombosis, tumors, the “ground-glass” patterns associated with pneumonia, apparent to skilled medical professionals. The target area is originated through one of any number of medical examination equipment including, but not limited to, X-rays, fluoroscopy, magnetic resonance, tomography, and ultrasound.

In view of the above, method 100 includes the step 110 of receiving one or more data sets associated with the target feature. With the birth of the digital technology, the output of the desired medical examination equipment can be reduced to a series of numerical representations—e.g., a data set associated with the target feature(s). Once reduced into the data domain, the target feature can now be enhanced for closer examination, study and detection, as desired. In one aspect of the disclosure, the at least one data set created for the target feature is resealed to enable for future processing benefits, such as, for example, enhanced speed of comparison and match identification.

With the receipt of data sets from the medical examination equipment, the method include the step 120 of creating a two-dimensional graphical representation of the data sets received in step 110. This step involved the formulation of at least one group of pixels or pixel grouping for each data set received from the target feature.

By way of merely an illustrative example, where the medical examination equipment used is a CT scan, the data sets received in step 120 will correspond with a series of two-dimensional cross-sectional views of the target feature. Here, each of these two-dimensional cross-sectional views can be reduced to a data set of floating numbers. For the purposes of illustration, each of these data sets may be floating numbers making up a single two-dimensional cross-sectional view as generated by the CT scan. This data set of floating numbers, for example, may comprises at least one group of pixels or pixel groupings. In practice, each two-dimensional cross-sectional view will likely include many pixel group, one or more of which include the target feature.

Method 100, with the pixel groups created for each two-dimensional cross-sectional view, may include the step 130 of a form of data compression. This approach takes into account considerations such gray-scale. Various compressions techniques are contemplated by this present disclosure include lossy or lossless compression for each pixel group. One such compression approach is vector attribute filtering. For the purpose of the present disclosure, attribute filtering use a criterion to remove or preserve connected components, or flat zones, based on their attributes. This typically involves removing objects, using an entire collection of pixel groupings data, that are similar enough to a given shape. Morphological attribute filters operate on pixel groupings based on properties or attributes of connected, or adjacent, pixel grouping components. Vector attribute filtering is a variant of morphological attribute filters in which the attribute on which filtering is based, is no longer a scalar but rather a vector. It should be noted that if a vector-attribute is a shape descriptor, the resulting granulometries filter an image based on a shape or shape family instead of one or more scalar values.

With vector attributes calculated for each of the pixel groups making up a data set from the medical examination source, the method includes a comparing step 140. In the context of the present disclosure, one aspect is to determine whether one or more pixel group, now characterized as vector attributes, can authenticated, and to what extent, with known data. The library of data may be formatted in any number of ways including uncompressed structure as well as lossy or lossless compression. In one aspect, the data library comprises vector attributes. It should however be noted that the methodologically and systematically, the vector attribute filtering of the data library can be performed on demand at the library or within the medical examination source performing method 100.

In one embodiment, the purpose comparison step 140 is to compare each pixel group with the data library of data to determine if there is or are known similarities between the target feature from the medical examination source and the pool of existing data. As noted herein, each pixel group from the target feature can be a vector attribute in one aspect of the disclosure. By this step, the medical professional may be more able to discern whether, for example, a static hypodermic, subdural and/or subcutaneous target feature from an exemplary CT scan slices taken of the specific area(s) of the body has an anomaly, such as a tumor, pneumonia or COVID-19, otherwise not discernable to the naked human eye, or otherwise occluded from view by a non-targeted feature(s).

As a consequence of performing the comparison step 140, method 100 then performs step 150 of selecting the highest match or matches between each vector attribute from the target feature and the data library. This step may be executed by various schema including but not limited to machine learning. In selecting the highest match or matches, step 150 scores or grades each match each vector attribute against from the target feature and the data library. In one aspect of the disclosure, a score threshold is utilized. This eliminates any match, including the highest match or matches, that fail to meet or exceed a threshold score.

In another embodiment of the present disclosure, comparing step 140 includes scanning each vector attribute filtered data from each pixel group of each data set of the target feature. By performing this scanning step, at least one max-tree of at least two dimensions from each vector attribute filtered data can be computed. As noted hereinabove, a max-tree is a hierarchical representation of at least one image forming the basis of a large family of morphological filters. Upon performing this calculating step, the at least one max-tree may then be compared with each vector attribute data in the library.

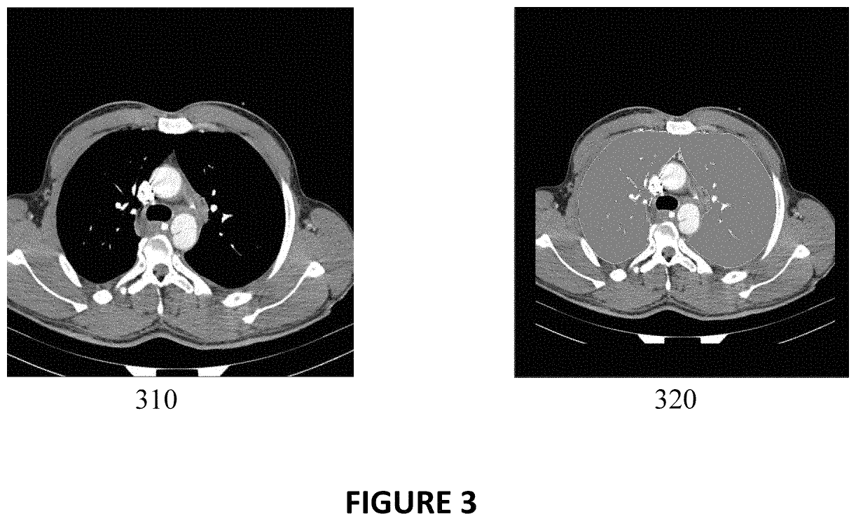

Referring to FIG. 3, another embodiment of the present disclosure is illustrated. Here, a medical system 200 is shown for examining target features with a data library. Medical system 200 includes a source 210 for generating at least one data set of the target feature. As noted herein, source 210 may include, but not be limited to X-rays, fluoroscopy, magnetic resonance, tomography, and ultrasound machines.

Medical system 200 further includes a computer processing tool 220. Tool 220 performs a variety of functions and may be realized in hardware, firmware or a combination thereof. In one embodiment, tool 220 includes machine learning capabilities.

Tool 220 creates at least one pixel grouping for each data set of the target feature. In one aspect of the present disclosure, tool 220 also rescales the at least one data set created for the target feature to enable for future processing benefits, such as, for example, enhanced speed of comparison and match identification. Further, tool may also perform lossy compression data, lossless compression data or vector attribute on each one pixel group from each data set of the target feature.

Tool 220 is electrical coupled with a data library 250 through data input line 230 and data output line 240. Through it electrical coupling with data library 250, tool 220 may compare the at least one pixel grouping with the data in data library 250 and may select one or more matching pixel groups between the target feature and the data library. It should be note that the data in data library 250 may include lossy compression data, lossless compression data or vector attribute data.

In selecting the highest match or matches, tool 220 may also score or grade each match each vector attribute against from the target feature and the data library. In one aspect of the disclosure, a score threshold is utilized. This eliminates any match, including the highest match or matches, that fail to meet or exceed a threshold score.

In one embodiment, tool 220 includes a display integrated therein for displaying the highest match or matches. It should be noted that the display need not be integrated with tool 220 and may be a stand-alone unit or part of some other system.

In another aspect of the present disclosure, tool 220 may also scans each vector attribute data from the least one pixel group and computes at least one max-tree of at least two dimensions from each vector attribute filtered data. Once completed, tool 220 may then compare the at least one max-tree with each vector attribute data in library 250.

Referring to FIG. 3, another aspect of the present disclosure is illustrated. In two dimensional scan 310, a cross sectional view is shown including a target feature. By comparison, two dimensional scan 320 shows a number of pixel groups.

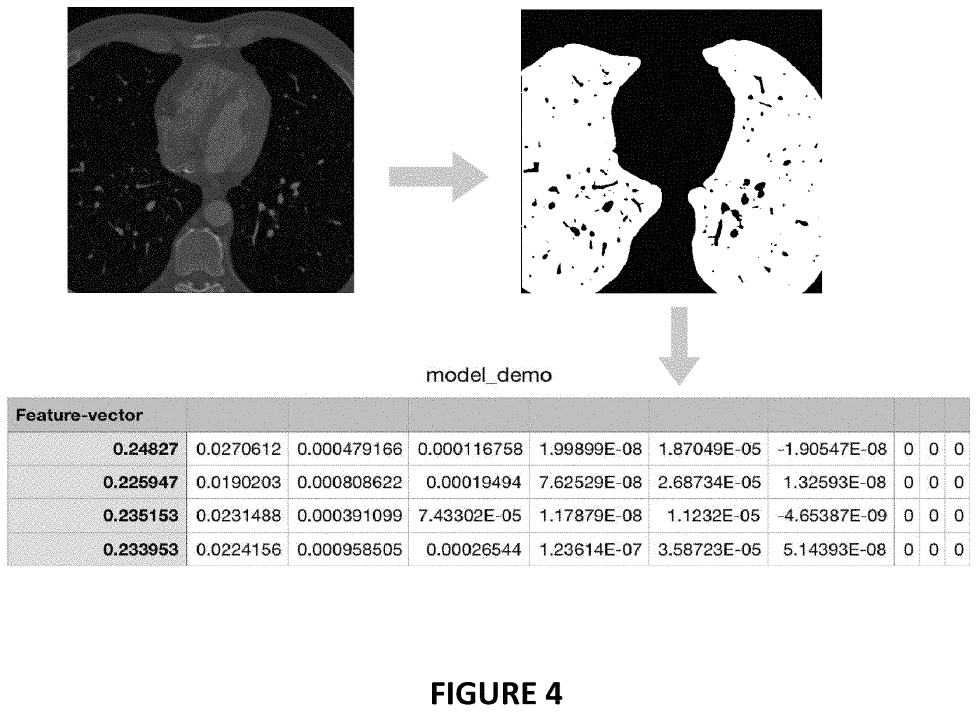

Referring to FIG. 4, yet another aspect of the present disclosure is depicted. In two dimensional scan 410, a cross sectional view is shown including a target feature. By comparison, two dimensional scan 420 shows a number of pixel groups. Feature vector table 430 illustrates the resultant conversion of pixel groups from scan 420 into vector attribute data.

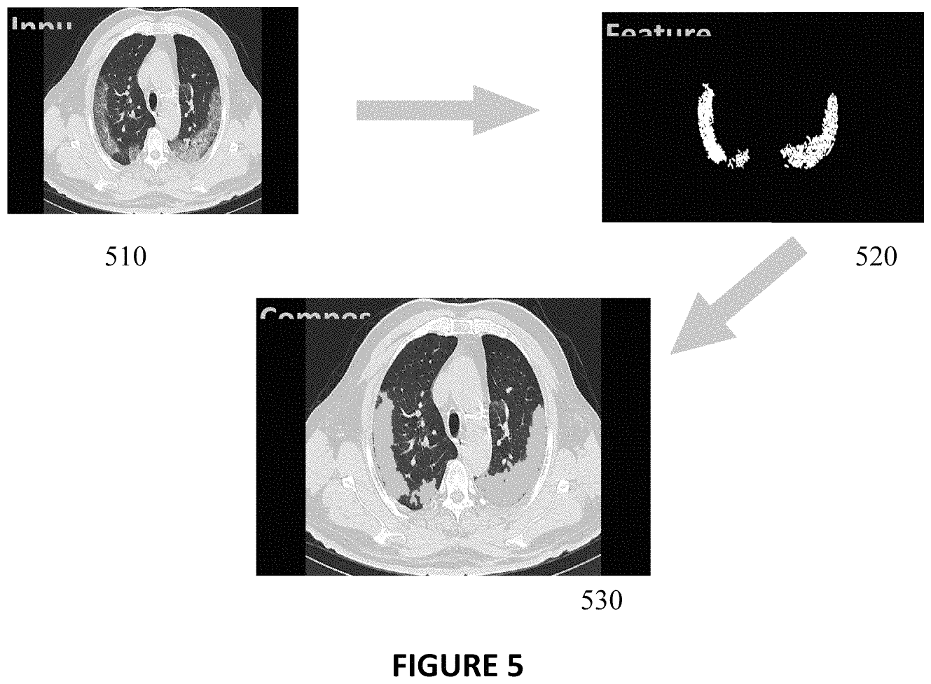

Referring to FIG. 5, still yet another aspect of the present disclosure is illustrated. In two dimensional scan 510, a cross sectional view is shown including a target feature. By comparison, scan 520 solely shows a number of pixel groups extracted from scan 510 as detailed herein. Scan 530 shows the pixel groups as highlighted in scan 520 super-positioned onto two dimensional scan 510.

It should be understood that the figures in the attachments, which highlight the structure, methodology, functionality and advantages of this disclosure, are presented for example purposes only. This disclosure is sufficiently flexible and configurable, such that it may be implemented in ways other than that shown in the accompanying figures.

What is claimed is:

1. A method for examining at least one target feature with medical examination equipment, the method of examining comprising the steps of:
receiving at least one data set of the target feature from the medical examination equipment;
creating at least one pixel grouping for each data set of the target feature;
comparing the at least one pixel grouping with a library of data; and
selecting at least one matching pixel group between the target feature and library of data.

2. The method of claim 1, further comprising the step:
resealing the at least one data set of the target feature from the medical examination equipment.

3. The method of claim 2, wherein the step of selecting at least one matching pixel group comprises:
scoring the at least one matching pixel group between each of the at least one pixel grouping with the library of data.

4. The method of claim 3, wherein the least one pixel group comprises:
at least one of lossy compression data, lossless compression data and vector attribute data.

5. The method of claim 4, where the data in the library of data comprises:
at least one of lossy compression data, lossless compression data and vector attribute data.

6. The method of claim 5, further comprising:
scanning each vector attribute data from the least one pixel group;
computing at least one max-tree of at least two dimensions from each vector attribute data; and
comparing the at least one max-tree with each vector attribute data in the library.

7. The method of claim 6, where the step of comparing is performed by a machine learning processing step.

8. A medical system for examining target features with a data library, the medical system comprising:
a source for generating at least one data set of the target feature;
a computer processing tool for creating at least one pixel grouping for each data set of the target feature;
comparing the at least one pixel grouping with a library of data;
selecting at least one matching pixel group between the target feature and the data library; and
a display for displaying the selected at least one matching pixel group.

9. The medical system of claim 8, wherein the computer processing tool further rescales the at least one data set created for the target feature.

10. The medical system of claim 9, wherein the computer processing tool scores the at least one matching pixel group between each of the at least one pixel grouping with the library of data.

11. The medical system of claim 10, wherein the computer processing tool performs on the at least one pixel group at least one lossy compression data, lossless compression data and vector attribute data.

12. The medical system of claim 11, where the data in the library of data comprises at least one of lossy compression data, lossless compression data and vector attribute data.

13. The medical system of claim 12, wherein the computer processing tool further
scans each vector attribute data from the least one pixel group;
computes at least one max-tree of at least two dimensions from each vector attribute data; and
compares the at least one max-tree with each vector attribute data in the library.

14. The medical system of claim 13, wherein the computer processing tool comprises a machine learning system.
